(19) United States
(12) Patent Application Publication
(10) Pub. No.: US 2023/0398126 A1
(43) Pub. Date: Dec. 14, 2023

(54) RIFT VALLEY FEVER SMALL MOLECULE TREATMENT

(71) Applicants: The Curators of the University of Missouri, Columbia, MO (US); University of Kansas, Lawrence, KS (US)

(72) Inventors: Wenjun MA, Columbia, MO (US); Heidi LIU, Columbia, MO (US); Anuradha ROY, Kansas City, MO (US)

(73) Assignees: The Curators of the University of Missouri, Columbia, MO (US); University of Kansas, Lawrence, KS (US)

(21) Appl. No.: 18/208,586

(22) Filed: Jun. 12, 2023

Related U.S. Application Data
(60) Provisional application No. 63/352,054, filed on Jun. 14, 2022.

(51) Int. Cl. A61K 31/554; A61K 31/551; A61K 31/497; A61K 31/4985; A61K 31/445; A61K 31/506; A61K 31/4162

(52) U.S. Cl. CPC A61K 31/554; A61K 31/551; A61K 31/497; A61K 31/4985; A61K 31/445; A61K 31/506; A61K 31/4162

(57) ABSTRACT

A method of treating and/or preventing Rift Valley fever, comprising administering an effective amount of a compound of formula (I), formula (II), formula (III), formula (IV), formula (V), formula (VI), formula (VII) or formula (VIII), or a salt thereof, to a subject need thereof; a pharmaceutical composition comprising a compound of formula (I), formula (II), formula (III), formula (IV), formula (V), formula (VI), formula (VII) or formula (VIII), or a salt thereof, and a pharmaceutically acceptable carrier; and a pharmaceutical composition comprising a compound selected from the group consisting of compound WMA-RV1, compound WMA-RV2, compound MA-RV3, compound WMA-RV4, compound WMA-RV5, compound WMA-RV6, compound WMA-RV7, or compound WMA-RV8, or a salt thereof, and a pharmaceutically acceptable carrier.

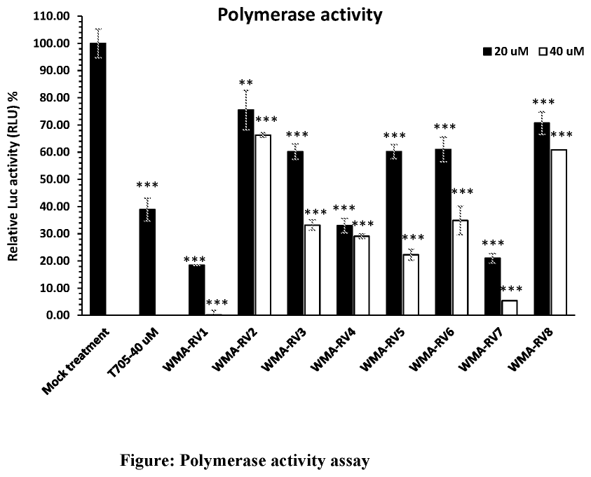

### CROSS-REFERENCE TO RELATED APPLICATIONS

This application claims the benefit of U.S. Provisional Application No. 63/352,054, filed Jun. 14, 2022, which is incorporated herein by reference in its entirety.

### STATEMENT REGARDING FEDERALLY SPONSORED RESEARCH OR DEVELOPMENT

This invention was made with government support under grant number R21 AI128377 awarded by the National Institutes of Health. The government has certain rights in the invention.

### BACKGROUND OF THE INVENTION

Rift Valley fever virus (hereinafter, “RVFV”) is an emerging, aerosol- and mosquito-borne human and veterinary pathogen that has caused large outbreaks of severe disease throughout Africa and more recently, in the Arabian Peninsula. Because RVFV is no longer restricted to African countries, it has raised concerns that the disease could spread worldwide (NPL-1). RVFV infection results in severe disease and abortion in cattle, sheep and goats, with 70-100% mortality in young animals (NPL-2). Human infections typically occur through infected mosquito bites or as the result of percutaneous/aerosol exposure during the slaughter of infected animals as well as via contact with aborted fetal materials (NPL-3). Normally, humans infected with RVFV have a self-limiting febrile disease and spontaneously recover; however, approximately 5% of patients will develop complications including hepatitis, encephalitis, and retinitis or a hemorrhagic syndrome with 10-20% fatality (NPL-4, NPL-5, NPL-6).

The US Centers for Disease Control and Prevention and the Department of Agriculture have classified RVFV as a Category A pathogen and an overlap select agent (NPL-7). Due to the high biocontainment facilities required to operate the virulent RVFV, limited laboratories have access to the virulent RVFV strains and perform efficacy testing of RVFV antivirals and vaccines. This limitation has hindered the development of antivirals and vaccines. Although there have been many studies on inactivated, live attenuated and molecular vaccines, there is still no fully licensed vaccine available for humans or animal use outside of endemic countries (NPL-8, NPL-9, NPL-10, NPL-11). The accidental or deliberate introduction of RVFV into non-endemic areas, such as the U.S., is a significant concern for the agriculture and public health because most non-endemic countries are not prepared for an introduction of RVFV. Once RVFV is introduced to the U.S., the virus will likely spread throughout the country much like the West Nile virus (NPL-11) because each region of the U.S. has the competent vector mosquito for RVFV (NPL-12, NPL-13, NPL-14, NPL-15). Despite the significant impact of the disease to the U.S. economy and public health, there are no FDA or USDA-approved therapeutic or prophylactic treatments available for infected or exposed humans.

Previous studies showed that several antivirals seem to be effective for RVFV (NPL-16, NPL-17, NPL-18, NPL-19, NPL-20, NPL-21, NPL-22), but current treatment options for RVFV-infected humans and animals are extremely limited. Ribavirin is the only licensed antiviral drug of all tested compounds and has been used to treat patients during past outbreaks. However, it has rather limited utility because it cannot go through the blood-brain barrier to reach the nervous system and has adverse side effects and concerns related to its potential to cause birth defects (NPL-23, NPL-8, NPL-24, NPL-25, NPL-26, NPL-28). Other “promising” compounds have not been investigated in detail. Questions regarding efficacy, safety, administration route and uptake and clearance of these antiviral compounds in animals also need to be addressed. In addition, no studies in natural hosts have been conducted. The lack of antivirals currently licensed and available to treat viral hemorrhagic fever infections, coupled with the ability of RNA viruses to mutate and develop resistance to drugs, highlights the urgent need for continuing identification and development of efficacious antiviral drugs against RVFV.

Like all other of bunyaviruses, RVFV is an enveloped negative-stranded RNA virus characterized by a tripartite genome composed of L, M and S segments (NPL-29). Phylogenetic analysis shows that the genetic diversity of all characterized strains of RVFV including the vaccine strain MP12 remains relatively small (NPL-9), suggesting an antiviral drug effective for one strain should be effective against all strains (NPL-17). Indeed, Ribavirin and 6-azauridine have been demonstrated to have antiviral activity on tested virulent RVFVs (NPL-17), and also inhibited MP12 vaccine and its divergents (NPL-30).

The present inventors previously developed a cell-based high-throughput assay with an average Z′-factor of 0.73 (NPL-30), indicating that it is suitable for screening a large amount of compounds against RVFV (NPL-31). The inventors also previously established a mouse model susceptible to infection with the MP12 vaccine strain which can be used to evaluate antiviral drugs in a BLS-2 facility (NPL-32).

By using their developed high-throughput assay and the mouse model and their understanding of the potential mechanisms of their inhibitory effects against RVFV, the inventors have now identified and evaluated effective compounds against RVFV for the treatment and prevention of Rift Valley fever.

### NON-PATENT REFERENCE LIST

NPL-1: Shoemaker, T., et al., 2002. Genetic analysis of viruses associated with emergence of Rift Valley fever in Saudi Arabia and Yemen, 2000-01. Emerg Infect Dis 8:1415-20.
NPL-2: Gerdes, G. H. 2004. Rift Valley fever. Rev Sci Tech 23:613-23.
NPL-3: Bird, B. H., et al., 2008. Rift valley fever virus lacking the NSs and NSm genes is highly attenuated, confers protective immunity from virulent virus challenge, and allows for differential identification of infected and vaccinated animals. J Virol 82:2681-91.
NPL-4: Madani, T. A., et al., 2003. Rift Valley fever epidemic in Saudi Arabia: epidemiological, clinical, and laboratory characteristics. Clin Infect Dis 37:1084-92.
NPL-5: McIntosh, B. M., et al., 1980. Rift Valley fever in humans in South Africa. S Afr Med J 58:803-6.
NPL-6: van Velden, D. J., et al., 1977. Rift Valley fever affecting humans in South Africa: a clinicopathological study. S Afr Med J 51:867-71.
NPL-7: Bird, B. H., et al., 2009. Rift Valley fever virus. J Am Vet Med Assoc 234:883-93.
NPL-8: Bouloy, M., et al., 2009. Reverse genetics technology for Rift Valley fever virus: current and future applications for the development of therapeutics and vaccines. Antiviral Res 84:101-18.
NPL-9: Ikegami, T. 2012. Molecular biology and genetic diversity of Rift Valley fever virus. Antiviral Res 95:293-310.
NPL-10: Ikegami, T., et al. 2004. [Rift Valley fever virus]. Uirusu 54:229-35.
NPL-11: Pepin, M., et al., 2010. Rift Valley fever virus(Bunyaviridae: Phlebovirus): an update on pathogenesis, molecular epidemiology, vectors, diagnostics and prevention. Vet Res 41:61.
NPL-12: Brubaker, J. F., et al., 1998. Effect of environmental temperature on the susceptibility of Culex pipiens (Diptera: Culicidae) to Rift Valley fever virus. J Med Entomol 35:918-21
NPL-13: House, J. A., et al., 1992. Rift Valley fever: present status and risk to the Western Hemisphere. Ann N Y Acad Sci 653:233-42.
NPL-14: Turell, M. J., 2008. Potential for North American mosquitoes to transmit Rift Valley fever virus. J Am Mosq Control Assoc 24:502-7.
NPL-15: Turell, M. J., et al., 1998. Susceptibility of selected strains of Australian mosquitoes (Diptera: Culicidae) to Rift Valley fever virus. J Med Entomol 35:132-5.
NPL-16: Canonico, P. G., et al., 1982. Antiviral efficacy of pyrazofurin against selected RNA viruses. Antiviral Res 2:331-7.
NPL-17: Garcia, S., et al., 2001. Quantitative real-time PCR detection of Rift Valley fever virus and its application to evaluation of antiviral compounds. J Clin Microbiol 39:4456-61.
NPL-18: Goebel, R. J., et al., 1982. Synthesis and antiviral activity of certain carbamoylpyrrolopyrimidine and pyrazolopyrimidine nucleosides. J Med Chem 25:1334-8.
NPL-19: Koehler, J. W., et al., 2013. A fusion-inhibiting peptide against Rift Valley fever virus inhibits multiple, diverse viruses. PLoS Negl Trop Dis 7:e2430.
NPL-20: Panchal, R. G., et al., 2012. Identification of an antioxidant small-molecule with broad-spectrum antiviral activity. Antiviral Res 93:23-9.
NPL-21: Peters, C. J., et al., 1986. Prophylaxis of Rift Valley fever with antiviral drugs, immune serum, an interferon inducer, and a macrophage activator. Antiviral Res 6:285-97.
NPL-22: Scharton, D., et al., 2014. Favipiravir (T-705) protects against peracute Rift Valley fever virus infection and reduces delayed-onset neurologic disease observed with ribavirin treatment. Antiviral Res 104:84-92.
NPL-23: Borio, L., et al., 2002. Hemorrhagic fever viruses as biological weapons: medical and public health management. JAMA 287:2391-405.
NPL-24: Kilgore, P. E., et al., 1997. Treatment of Bolivian hemorrhagic fever with intravenous ribavirin. Clin Infect Dis 24:718-22.
NPL-25: McCormick, J. B., et al., 1986. Lassa fever. Effective therapy with ribavirin. N Engl J Med 314:20-6.
NPL-26: Monath, T. P. 2008. Treatment of yellow fever. Antiviral Res 78:116-24.
NPL-27: Sidwell, R. W., et al., 1988. Effects of ribamidine, a 3-carboxamidine derivative of ribavirin, on experimentally induced Phlebovirus infections. Antiviral Res 10:193-207.
NPL-29: Bouloy, M., et al., 2010. Molecular biology of rift valley Fever virus. Open Virol J 4:8-14.
NPL-30: Lang, Y., et al., 2019. Identification and evaluation of antivirals for Rift Valley fever virus. Vet Microbiol 230:110-116.
NPL-31: Zhang, J., et al., 1999. A simple statistical parameter for use in evaluation and validation of high throughput screening assays. Journal of biomolecular screening 4:67-73.
NPL-32: Lang, Y., et al., 2016. Mouse model for the Rift Valley fever virus MP12 strain infection. Vet Microbiol 195:70-77.

### BRIEF SUMMARY OF THE INVENTION

The inventors have identified and evaluated effective compounds against RVFV by using their developed high-throughput assay and the mouse model, and their understanding of the potential mechanisms of their inhibitory effects of identified candidates against RVFV. The present application discloses a method of treating and/or preventing Rift Valley fever (hereinafter, “RVF” or “Rift Valley fever”) comprising the administration of any one of these effective compounds or a salt thereof to a subject, such as a human or animal. The present application further discloses pharmaceutical compositions comprising any one of these compounds or a salt thereof and a pharmaceutically acceptable carrier. The compounds are expected to have the ability to cross the blood-brain barrier.

More particularly, the invention is described as follows.
[1] A method of treating and/or preventing Rift Valley fever, comprising administering an effective amount of a compound of formula (I),

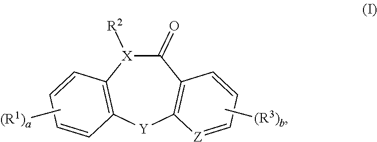

or a salt thereof,
wherein:
X is S, N and O;
Y is S, N and O;
Z is C(R^{3a}) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R² is H, C₁₋₈ alkyl, C₂₋₈ alkenyl, C₂₋₈ alkynyl, C₃₋₇ cycloalkyl, C₁₋₈ alkoxy, halogen, or halo-substituted C₁₋₈ alkoxy, wherein the C₁₋₈ alkyl, C₂₋₈ alkenyl and C₂₋₈ alkynyl are optionally substituted with halogen, C₁₋₈ alkyl, hydroxy, oxo, C₁₋₈ alkoxy, C₃₋₇ cycloalkyl, cyano or phenyl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R^{3a} is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;

R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 1, 2, 3 or 4;
b is 0, 1, 2 or 3; and
m is 1 or 2;
   to a subject in need thereof.
[2] The method according to [1], wherein:
X is N;
Y is S or O;
Z is N;
R¹ is independently H, C₁₋₈ alkyl, C(═O)N(R⁴)(R⁵) or C(═O)C(R⁴)(R⁵), wherein the C₁₋₈ alkyl is optionally substituted with substituent(s) independently selected from the group consisting of halogen, C₁₋₄ alkyl and hydroxy;
R⁴ is H or C₁₋₆ alkyl; and
R⁵ is C₁₋₆ alkyl, C₃₋₇ cycloalkyl, phenyl or 5- to 6-membered heteroaryl, wherein the C₁₋₆ alkyl, C₃₋₇ cycloalkyl, phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl.
[3] The method according to [1], wherein:
X is N;
Y is S;
Z is N;
R¹ is C(═O)NR⁴R⁵;
R⁴ is H or C₁₋₄ alkyl;
R⁵ is phenyl or 5- to 6-membered heteroaryl, wherein the phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl;
a is 1; and
b is 0.
[4] The method according to [1], wherein the compound of formula (I) is compound WMA-RV1,

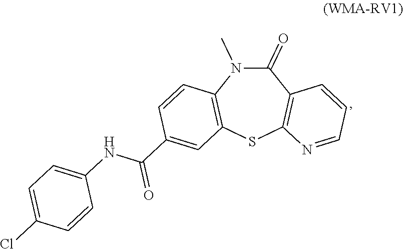 [SMILES: CN1C(=O)c2cccnc2Sc2cc(C(=O)Nc3ccc(Cl)cc3)ccc21]

or a salt thereof.
[5] A method of treating and/or preventing Rift Valley fever, comprising administering an effective amount of a compound of formula (II),

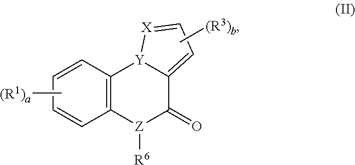

or a salt thereof,
wherein:
X is S, N or O;
Y is S, N or O;
Z is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁶ is H, C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
a is 0, 1, 2, 3 or 4;
b is 0, 1 or 2; and
m is 1 or 2;
   to a subject in need thereof.
[6] The method according to [5], wherein:
X is N;
Y is N;
Z is N;
R¹ is independently H, C₁₋₈ alkyl, C(═O)N(R⁴)(R⁵) or C(═O)C(R⁴)(R⁵);
R⁶ is C₁₋₈ alkyl, C₂₋₈ alkenyl or C₂₋₈ alkynyl, wherein the C₁₋₈ alkyl, C₂₋₈ alkenyl or C₂₋₈ alkynyl is substituted with optionally substituted C₃₋₇ cycloalkyl, optionally substituted C₆₋₁₀ aryl or optionally substituted 5- to 10-membered heteroaryl, wherein the optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₁₋₈ alkoxy and halogen;
a is 1; and
b is 0.
[7] The method according to [5], wherein:
X is N;
Y is N;
Z is N;
R¹ is C(═O)N(R⁴)(R⁵) or C(═O)C(R⁴)(R⁵);
R⁴ is H or C₁₋₈ alkyl;
R⁵ is H or C₁₋₈ alkyl;
R⁶ is C₁ alkyl, which is substituted with optionally substituted C₃₋₇ cycloalkyl, optionally substituted phenyl or optionally substituted 5- or 6-membered heteroaryl, wherein the optionally substituted C₃₋₇ cycloalkyl, optionally substituted phenyl and optionally substituted 5- or 6-membered heteroaryl is substituted with substituent(s) independently selected from the group consisting of C₁₋₄ alkyl, halogen and halogenated-C₁₋₄ alkyl;
a is 1; and
b is 0.
[8] The method according to [5], wherein the compound of formula (II) is compound WMA-RV2,

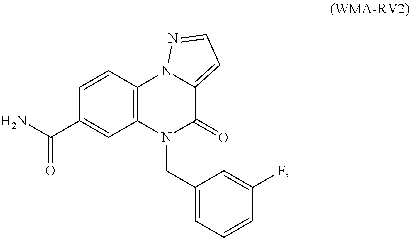 [SMILES: NC(=O)c1ccc2c(c1)n(Cc1cccc(F)c1)c(=O)c1ccnn12]

or a salt thereof.
[9] A method of treating and/or preventing Rift Valley fever, comprising administering an effective amount of a compound of formula (III),

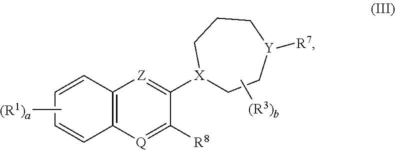

or a salt thereof,
wherein:
X is S, N or O;
Y is S, N or O;
Z is S, N or O;
Q is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁷ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁸ is optionally substituted C₃₋₇ cycloalkyl, optionally substituted C₆₋₁₀ aryl, optionally substituted 4- to 8- membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 0, 1, 2, 3 or 4;
b is 0, 1, 2, 3, 4 or 5; and
m is 1 or 2;
   to a subject in need thereof.
[10] The method according to [9], wherein:
X is N;
Y is N;
Z is N;
Q is N;
R¹ is independently H, C₁₋₈ alkyl or halogen;
R³ is independently H, C₁₋₈ alkyl or halogen;
R⁷ is H, C₁₋₆ alkyl, C(═O)N(R⁴)(R⁵) or C(═O)C(R⁴)(R⁵);
R⁴ is H, C₁₋₆ alkyl or halogen;
R⁵ is H, C₁₋₆ alkyl, or halogen;
R⁸ is optionally substituted C₆₋₁₀ aryl or optionally substituted 5- to 6-membered heteroaryl;
a is 0 or 1; and
b is 0 or 1.
[11] The method according to [9], wherein:
X is N;
Y is N;
Z is N;
Q is N;
R¹ is independently H or C₁₋₄ alkyl;
R⁷ is C(═O)C(R⁴)(R⁵);
R⁴ is C₁₋₄ alkyl;
R⁵ is halogen;
R⁸ is phenyl, which is optionally substituted with halogen or C₁₋₄ alkyl;
a is 0 or 1; and
b is 0.
[12] The method according to [9], wherein the compound of formula (III) is compound WMA-RV3,

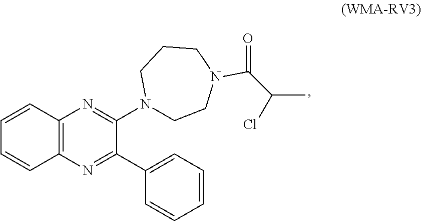 [SMILES: CC(Cl)C(=O)N1CCCN(c2nc3ccccc3nc2-c2ccccc2)CC1]

or a salt thereof.
[13] A method of treating and/or preventing Rift Valley fever, comprising administering an effective amount of a compound of formula (IV),

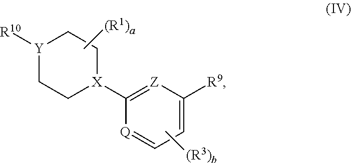

or a salt thereof,
wherein:
X is S, N or O;
Y is S, N or O;
Z is S, N or O;
Q is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁹ is optionally substituted C₃₋₇ cycloalkyl, optionally substituted C₆₋₁₀ aryl, optionally substituted 5- to 10-membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano and optionally substituted C₆₋₁₀ aryl;

R¹⁰ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
a is 0, 1, 2, 3 or 4;
b is 0, 1 or 2; and
m is 1 or 2;
   to a subject in need thereof.
[14] The method according to [13], wherein
X is N;
Y is N;
Z is N;
Q is N;
R¹ is independently H, C₁₋₈ alkyl or halogen;
R³ is independently H, C₁₋₈ alkyl or halogen;
R⁹ is optionally substituted C₆₋₁₀ aryl or optionally substituted 5- to 6-membered heteroaryl, wherein the C₆₋₁₀ aryl or 5- to 6-membered heteroaryl is optionally substituted with substituent(s) independently selected from the group consisting of halogen, optionally substituted

C₁₋₈ alkyl, hydroxy, optionally substituted C₁₋₈ alkoxy, optionally substituted C₃₋₇ cycloalkyl and cyano;
R¹⁰ is C₁₋₈ alkyl, C₃₋₇ cycloalkyl, C₁₋₈ alkoxy, C(═O)N(R⁴)(R⁵) or C(═O)C(R⁴)(R⁵); R⁴ is H, C₁₋₈ alkyl, C₃₋₇ cycloalkyl, halogen or phenyl, wherein the C₁₋₈ alkyl, C₃₋₇ cycloalkyl or phenyl are optionally substituted with halogen, C₁₋₄ alkyl or hydroxy;
R⁵ is H, C₁₋₈ alkyl, C₃₋₇ cycloalkyl, halogen or phenyl, wherein the C₁₋₈ alkyl, C₃₋₇ cycloalkyl or phenyl are optionally substituted with halogen, C₁₋₄ alkyl or hydroxy;
a is 0 or 1; and
b is 0 or 1.
[15] The method according to [13], wherein
X is N;
Y is N;
Z is N;
Q is N;
R¹ is independently H, C₁₋₄ alkyl or halogen;
R³ is independently H, C₁₋₄ alkyl or halogen;
R⁹ is phenyl substituted with C₁₋₈ alkyl, which is optionally substituted with C₁₋₈ alkyl, C₁₋₈ alkoxy or halogen;
R¹⁰ is C(═O)N(R⁴)(R⁵) or C(═O)C(R⁴)(R⁵);
R⁴ is H, C₁₋₈ alkyl or halogen;
R⁵ is H, C₁₋₈ alkyl or halogen;
a is 0 or 1; and
b is 0 or 1.
[16] The method according to [13], wherein the compound of formula (IV) is compound WMA-RV4,

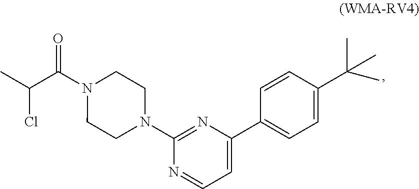 [SMILES: CC(Cl)C(=O)N1CCN(c2nccc(-c3ccc(C(C)(C)C)cc3)n2)CC1]

or a salt thereof.
[17] A method of treating and/or preventing Rift Valley fever, comprising administering an effective amount of a compound of formula (V),

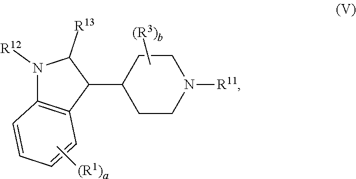

or a salt thereof,
wherein:
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R¹¹ is —C₁₋₄alkyl-S(O)ₙ—N(R⁴)(R⁵)-R¹⁴—S(O)ₙ—C₁₋₄alkyl—N(R⁴)(R⁵)-R¹⁴, —S(O)ₙ—C₁₋₄alkyl-C(═O)C(R⁴)(R⁵)-R¹⁴, —S(O)ₙ—C(═O)C(R⁴)(R⁵)—R¹⁴, —S(O)ₙ—R¹⁴, —S(O)ₙ—C₁₋₄alkyl-R¹⁴ or -C₁₋₄alkyl-S(O)ₙ—C₁₋₄alkyl-R¹⁴;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle or 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl;
R¹² is H, C₁₋₈ alkyl, C₃₋₇ cycloalkyl, halogen or C₆₋₁₀ aryl, wherein the C₁₋₈ alkyl, C₃₋₇ cycloalkyl C₆₋₁₀ aryl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R¹³ is H, C₁₋₈ alkyl or halogen;
a is 0, 1, 2, 3 or 4;
b is 0, 1, 2, 3 or 4;
m is 1 or 2; and
n is 1 or 2;
   to a subject in need thereof.
[18] The method according to [17], wherein:
R¹ is C₁₋₄ alkyl;
R¹² is H or C₁₋₄ alkyl;
R¹³ is H or C₁₋₄ alkyl;
R¹¹ is —S(O)ₙ—R¹⁴ , —S(O)ₙ—C₁₋₄alkyl-R¹⁴ or -C₁₋₄alkyl-S(O)ₙ—C₁₋₄alkyl-R¹⁴;
R¹⁴ is C₆₋₁₀ aryl or 5- to 6-membered heteroaryl, wherein the C₆₋₁₀ aryl and 5- to 6-membered heteroaryl are optionally substituted with substituent(s) independently selected from the group consisting of halogen, C₁₋₄ alkyl, hydroxy, C₁₋₆ alkoxy, cyano, amino and phenyl;
a is 1, 2 or 3;

b is 0; and
n is 2.
[19] The method according to [17], wherein the compound of formula (V) is a compound of formula (Va),

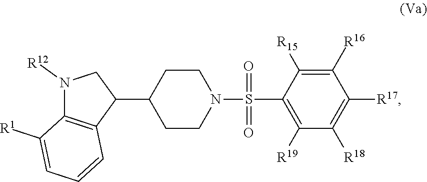

or a salt thereof,
wherein:
R¹ is C₁₋₄ alkyl;
R¹² is H or C₁₋₄ alkyl;
R¹⁵, R¹⁶, R¹⁷ , R¹⁸ and R¹⁹ are each independently H, C₁₋₆ alkyl, halogen, hydroxy, C₁₋₆ alkoxy, cyano, amino or phenyl.
[20] The method according to [17], wherein the compound of formula (V) is compound WMA-RV5,

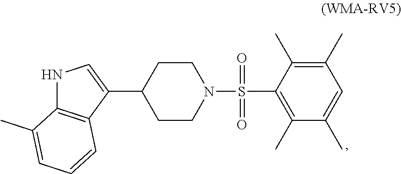

or a salt thereof.
[21] A method of treating and/or preventing Rift Valley fever, comprising administering an effective amount of a compound of formula (VI),

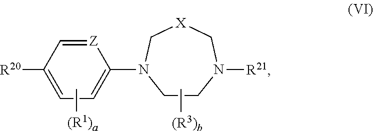

or a salt thereof,
wherein:
X is C(R²²)(R²³) or C(═O);
Z is C(R²⁴) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈alkoxy, C₃₋₇cycloalkyl, cyano and C₆₋₁₀ aryl;
R²⁰ is C(═O)N(R⁴)(R⁵), C(═O)C(R⁴)(R⁵), C₁₋₄alkylC(═O)—N(R⁴)(R⁵), N(R⁴) c(═O)—R¹⁴ or N(R⁴)C(═O)-C₁₋₄alkyl-R¹⁴ , wherein the C₁₋₄alkyl is optionally substituted with halogen, C₁₋₄ alkyl or C₁₋₄ alkoxy;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and phenyl;
R²¹ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
R²² is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²³ is H, C₁₋₈ alkyl, C₂₋₈ alkenyl, C₂₋₈ alkynyl, C₃₋₇ cylcoalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
a is 0, 1, 2 or 3;
b is 0, 1, 2, 3 or 4; and
m is 1 or 2;
   to a subject in need thereof.
[22] The method according to [21], wherein:
X is C(R²²)(R²³);
Z is N;
R²⁰ is N(R⁴)C(═O)—R¹⁴ N(R⁴)C(═O)—C₁₋₄alkyl-R¹⁴;
R⁴ is H or C₁₋₄ alkyl;
R¹⁴ is C₆₋₁₀ aryl or 5- to 10-membered heteroaryl, wherein the C₆₋₁₀ aryl and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and amino;
R²¹ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
b is 0 or 1.
[23] The method according to [21], wherein:
X is C(R²²)(R²³);
R²² is H;
R²³ is H:
Z is N;
R²⁰ is N(R⁴)C(═O)—R¹⁴;
R⁴ is H;
R¹⁴ is phenyl, which is optionally substituted with substituent(s) independently selected from the group consisting of halogen, C₁₋₄ alkyl, hydroxy, and C₁₋₄ alkoxy; and
R²¹ is C₁ alkyl substituted with phenyl, wherein the phenyl is optionally substituted with substituent(s) independently selected from the group consisting of halogen, C₁₋₄ alkyl, hydroxy, and C₁₋₄ alkoxy.
[24] The method according to [21], wherein the compound of formula (VI) is compound WMA-RV6,

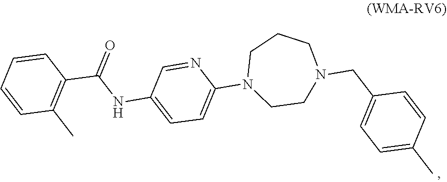 [SMILES: Cc1ccc(CN2CCCN(c3ccc(NC(=O)c4ccccc4C)cn3)CC2)cc1]

or a salt thereof.
[25] A method of treating and/or preventing Rift Valley fever, comprising administering an effective amount of a compound of formula (VII),

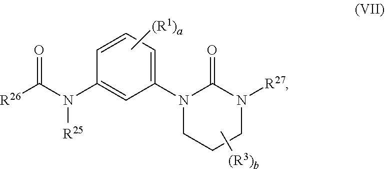

or a salt thereof,
wherein:
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R²⁵ is H, C₁₋₈ alkyl or C(═O)C(R⁴)(R⁵);
R²⁶ is H, C₁₋₈ alkyl, C₃₋₇ cycloalkyl, C₆₋₁₀ aryl, 5- to 10-membered heterocycle or 5- to 10-membered heteroaryl, wherein the C₁₋₈ alkyl, C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁷ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
a is 0, 1, 2, 3 or 4;
b is 0, 1, 2 or 3; and
m is 1 or 2;
   to a subject in need thereof.
[26] The method according to [25], wherein:
R²⁵ is H or C₁₋₄ alkyl;
R²⁶ is a 5- to 6-membered heterocycle or 5- to 6-membered heteroaryl, which is optionally substituted with substituent(s) independently selected from the group consisting of halogen, C₁₋₄ alkyl, hydroxy, oxo, C₁₋₄ alkoxy, C₃₋₇ cycloclkyl, cyano and C₆₋₁₀ aryl;
R²⁷ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
b is 0 or 1.
[27] The method according to [25], wherein:
R²⁵ is H;
R²⁶ is a 5- to 6-membered heterocycle, which is optionally substituted with halogen, C₁₋₄ alkyl, hydroxy, oxo, cyano, C₁₋₄ alkoxy or phenyl;
R²⁷ is C₁ alkyl substituted with phenyl, wherein the phenyl is optionally substituted with 1 to 5 substituents independently selected from the group consisting of halogen, C₁₋₄ alkyl, hydroxy and C₁₋₄ alkoxy;
a is 0; and
b is 0.
[28] The method according to [25], wherein the compound of formula (VII) is compound WMA-RV7,

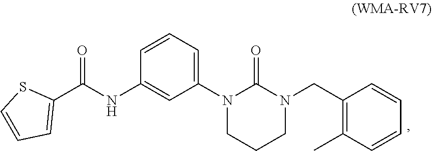 [SMILES: Cc1ccccc1CN1CCCN(c2cccc(NC(=O)c3cccs3)c2)C1=O]

or a salt thereof.
[29] A method of treating and/or preventing Rift Valley fever, comprising administering an effective amount of a compound of formula (VIII),

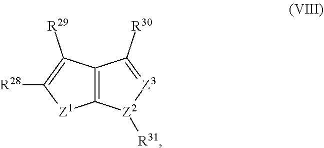

or a salt thereof,
wherein:
Z¹ is S, N or O;
Z² is S, N or O;
Z³ is S, N or O;
R²⁸ is C(═O)N(R⁴)(R⁵), C(═O)C(R⁴)(R⁵), C₁₋₄alkylC(═O)—N(R⁴)(R⁵), N(R⁴)C(═O)—R¹⁴ or N(R⁴)C(═O)—C₁₋₄alkyl-R¹⁴ , wherein the C₁₋₄alkyl is optionally substituted with halogen or C₁₋₄ alkyl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle or 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl;
R²⁹ is H, C₁₋₈ alkyl, C₃₋₇ cycloalkyl or phenyl, wherein the C₁₋₈ alkyl, C₃₋₇ cycloalkyl and phenyl are optionally substituted with halogen, C₁₋₄ alkyl, hydroxy, oxo, C₁₋₄ alkoxy, C₃₋₇ cycloalkyl or cyano;
R³⁰ is H, C₁₋₈ alkyl, C₃₋₇ cycloalkyl or C₆₋₁₀ aryl, wherein the C₁₋₈ alkyl, C₃₋₇ cycloalkyl and C₆₋₁₀ aryl are optionally substituted with halogen, C₁₋₄ alkyl, hydroxy, oxo, C₁₋₄ alkoxy, C₃₋₇ cycloalkyl or cyano; and
R³¹ is optionally substituted C₃₋₇ cycloalkyl, optionally substituted C₆₋₁₀ aryl, optionally substituted 5- to 10-membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl,
to a subject in need thereof.
[30] The method according to [29], wherein:
Z¹ is S or O;
Z² is N;
Z³ is N;
R²⁸ is C(═O)N(R⁴)(R⁵) or C₁₋₄alkylC(═O)—N(R⁴)(R⁵), wherein the C₁₋₄alkyl is optionally substituted with halogen or C₁₋₄ alkyl;
R⁴ is H or C₁₋₄ alkyl, wherein the C₁₋₄ alkyl is optionally substituted with halogen or C₁₋₄ alkyl;
R⁵ is H or C₁₋₄ alkyl, wherein the C₁₋₄ alkyl is optionally substituted with halogen or C₁₋₄ alkyl;
R²⁹ is H or C₁₋₄ alkyl, wherein the C₁₋₄ alkyl is optionally substituted with halogen or C₁₋₄ alkyl;
R³⁰ is H or C₁₋₄ alkyl, wherein the C₁₋₄ alkyl is optionally substituted with halogen or C₁₋₄ alkyl; and
R³¹ is optionally substituted phenyl or optionally substituted 5- to 6-membered heteroaryl, wherein the phenyl and 5- to 6-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl.
[31] The method according to [29], wherein:
Z¹ is S;
Z² is N;
Z³ is N;
R²⁸ is C(═O)N(R⁴)(R⁵);
R⁴ is H;
R⁵ is C₁₋₄ alkyl, wherein the C₁₋₄ alkyl is optionally substituted with halogen or C₁₋₄ alkyl;
R²⁹ is H;
R³⁰ is C₁₋₄ alkyl, wherein the C₁₋₄ alkyl is optionally substituted with halogen or C₁₋₄ alkyl; and
R³¹ is phenyl.
[32] The method according to [29], wherein the compound of formula (VIII) is

compound WMA-RV8,

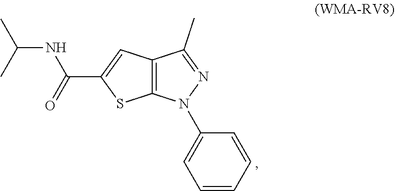 [SMILES: Cc1nn(-c2ccccc2)c2sc(C(=O)NC(C)C)cc12]

or a salt thereof.
[33] A method of treating and/or preventing Rift Valley Fever, comprising administering an effective amount of at least one compound selected from the group consisting of:

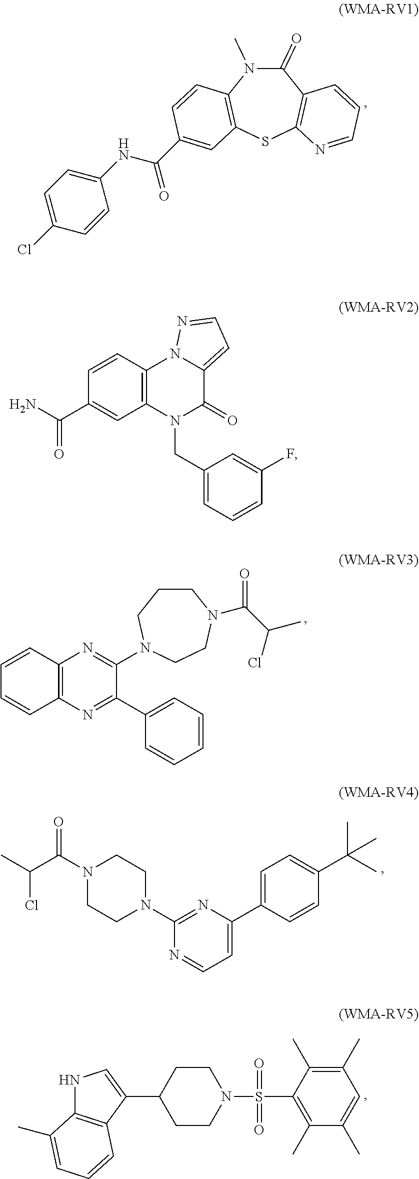

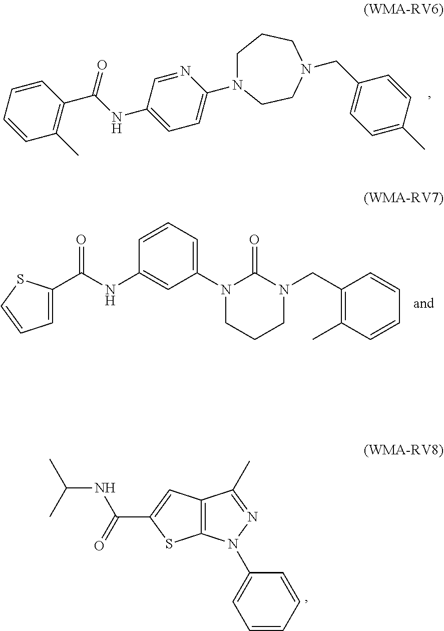

or a salt thereof,
to a subject in need thereof.
[34] A pharmaceutical composition comprising:
   a compound of formula (I),

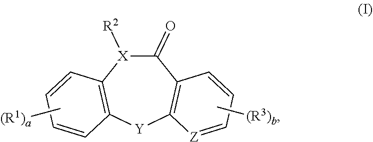

or a salt thereof,
wherein:
X is S, N and O;
Y is S, N and O;
Z is C(R^{3a}) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R² is H, C₁₋₈ alkyl, C₂₋₈ alkenyl, C₂₋₈ alkynyl, C₃₋₇ cycloalkyl, C₁₋₈ alkoxy, halogen, or halo-substituted C₁₋₈ alkoxy, wherein the C₁₋₈ alkyl, C₂₋₈ alkenyl and C₂₋₈ alkynyl are optionally substituted with halogen, C₁₋₈ alkyl, hydroxy, oxo, C₁₋₈ alkoxy, C₃₋₇ cycloalkyl, cyano or phenyl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R^{3a} is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 1, 2, 3 or 4;
b is 0, 1, 2 or 3; and
m is 1 or 2; and
   a pharmaceutically acceptable carrier.
[35] The pharmaceutical composition according to [34], wherein:
X is N;
Y is S or O;
Z is N;
R¹ is independently H, C₁₋₈ alkyl, C(═O)N(R⁴)(R⁵) or C(═O)C(R⁴)(R⁵), wherein the C₁₋₈ alkyl is optionally substituted with substituent(s) independently selected from the group consisting of halogen, C₁₋₄ alkyl and hydroxy;
R⁴ is H or C₁₋₆ alkyl; and
R⁵ is C₁₋₆ alkyl, C₃₋₇ cycloalkyl, phenyl or 5- to 6-membered heteroaryl, wherein the C₁₋₆ alkyl, C₃₋₇ cycloalkyl, phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl.
[36] The pharmaceutical composition according to [34], wherein:
X is N;
Y is S;
Z is N;
R¹ is C(═O)NR⁴ R⁵;
R⁴ is H or C₁₋₄ alkyl;
R⁵ is phenyl or 5- to 6-membered heteroaryl, wherein the phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl;
a is 1; and
b is 0.
[37] The pharmaceutical composition according to [34], wherein the compound of formula (I) is compound WMA-RV1,

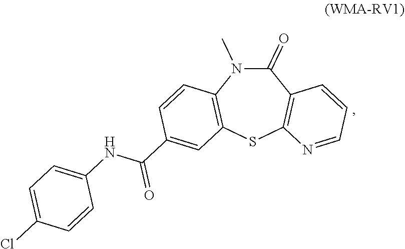 [SMILES: CN1C(=O)c2cccnc2Sc2cc(C(=O)Nc3ccc(Cl)cc3)ccc21]

or a salt thereof.
[38] A pharmaceutical composition comprising:
   a compound of formula (II),

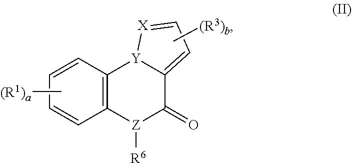

or a salt thereof,
wherein:
X is S, N or O;
Y is S, N or O;
Z is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁶ is H, C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
a is 0, 1, 2, 3 or 4;
b is 0, 1 or 2; and
m is 1 or 2; and
   a pharmaceutically acceptable carrier.
[39] The pharmaceutical composition according to [38], wherein:
X is N;
Y is N;
Z is N;
R¹ is independently H, C₁₋₈ alkyl, C(═O)N(R⁴)(R⁵) or C(═O)C(R⁴)(R⁵);
R⁶ is C₁₋₈ alkyl, C₂₋₈ alkenyl or C₂₋₈ alkynyl, wherein the C₁₋₈ alkyl, C₂₋₈ alkenyl or C₂₋₈ alkynyl is substituted with optionally substituted C₃₋₇ cycloalkyl, optionally substituted C₆₋₁₀ aryl or optionally substituted 5- to 10-membered heteroaryl, wherein the optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₁₋₈ alkoxy and halogen;
a is 1; and
b is 0.
[40] The pharmaceutical composition according to [38], wherein:
X is N;
Y is N;
Z is N;
R¹ is C(═O)N(R⁴)(R⁵) or C(═O)C(R⁴)(R⁵);
R⁴ is H or C₁₋₈ alkyl;
R⁵ is H or C₁₋₈ alkyl;
R⁶ is C₁ alkyl, which is substituted with optionally substituted C₃₋₇ cycloalkyl, optionally substituted phenyl or optionally substituted 5- or 6-membered heteroaryl, wherein the optionally substituted C₃₋₇ cycloalkyl, optionally substituted phenyl and optionally substituted 5- or 6-membered heteroaryl is substituted with substituent(s) independently selected from the group consisting of C₁₋₄ alkyl, halogen and halogenated-C₁₋₄ alkyl;
a is 1; and
b is 0.
[41] The pharmaceutical composition according to [38], wherein the compound of formula (II) is compound WMA-RV2,

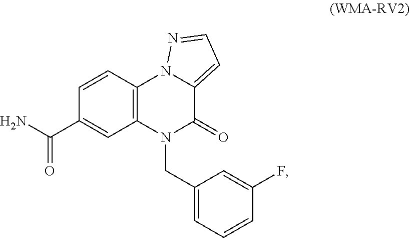 [SMILES: NC(=O)c1ccc2c(c1)n(Cc1cccc(F)c1)c(=O)c1ccnn12]

or a salt thereof.
[42] A pharmaceutical composition comprising:
   a compound of formula (III),

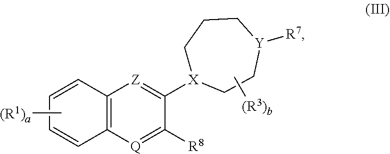

or a salt thereof,
wherein:
X is S, N or O;
Y is S, N or O;
Z is S, N or O;
Q is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁷ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁸ is optionally substituted C₃₋₇ cycloalkyl, optionally substituted C₆₋₁₀ aryl, optionally substituted 4- to 8- membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 0, 1, 2, 3 or 4;
b is 0, 1, 2, 3, 4 or 5; and
m is 1 or 2; and
   a pharmaceutically acceptable carrier.
[43] The pharmaceutical composition according to [42], wherein:
X is N;
Y is N;
Z is N;
Q is N;
R¹ is independently H, C₁₋₈ alkyl or halogen;
R³ is independently H, C₁₋₈ alkyl or halogen;
R⁷ is H, C₁₋₆ alkyl, C(═O)N(R⁴)(R⁵) or C(═O)C(R⁴)(R⁵);
R⁴ is H, C₁₋₆ alkyl or halogen;
R⁵ is H, C₁₋₆ alkyl, or halogen;
R⁸ is optionally substituted C₆₋₁₀ aryl or optionally substituted 5- to 6-membered heteroaryl;
a is 0 or 1; and
b is 0 or 1.
[44] The pharmaceutical composition according to [42], wherein:
X is N;
Y is N;
Z is N;
Q is N;
R¹ is independently H or C₁₋₄ alkyl;
R⁷ is C(═O)C(R⁴)(R⁵);
R⁴ is C₁₋₄ alkyl;
R⁵ is halogen;
R⁸ is phenyl, which is optionally substituted with halogen or C₁₋₄ alkyl;
a is 0 or 1; and
b is 0.
[45] The pharmaceutical composition according to [42], wherein the compound of formula (III) is compound WMA-RV3,

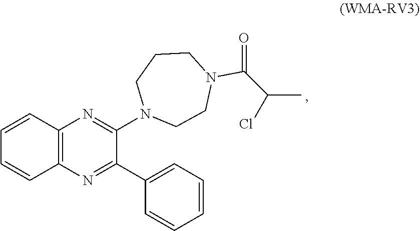 [SMILES: CC(Cl)C(=O)N1CCCN(c2nc3ccccc3nc2-c2ccccc2)CC1]

or a salt thereof.
[46] A pharmaceutical composition comprising:
   a compound of formula (IV),

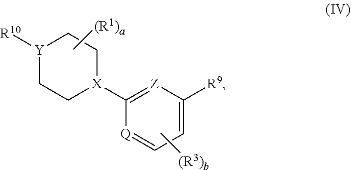

or a salt thereof,
wherein:
X is S, N or O;
Y is S, N or O;
Z is S, N or O;
Q is S, N or O;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R⁹ is optionally substituted C₃₋₇ cycloalkyl, optionally substituted C₆₋₁₀ aryl, optionally substituted 5- to 10-membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano and optionally substituted C₆₋₁₀ aryl;
R¹⁰ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
a is 0, 1, 2, 3 or 4;
b is 0, 1 or 2; and
m is 1 or 2; and
   a pharmaceutically acceptable carrier.
[47] The pharmaceutical composition according to [46], wherein:
X is N;
Y is N;
Z is N;
Q is N;
R¹ is independently H, C₁₋₈ alkyl or halogen;
R³ is independently H, C₁₋₈ alkyl or halogen;
R⁹ is optionally substituted C₆₋₁₀ aryl or optionally substituted 5- to 6-membered heteroaryl, wherein the C₆₋₁₀ aryl or 5- to 6-membered heteroaryl is optionally substituted with substituent(s) independently selected from the group consisting of halogen, optionally substituted C₁₋₈ alkyl, hydroxy, optionally substituted C₁₋₈ alkoxy, optionally substituted C₃₋₇ cycloalkyl and cyano;
R¹⁰ is C₁₋₈ alkyl, C₃₋₇ cycloalkyl, C₁₋₈ alkoxy, C(═O)N(R⁴)(R⁵) or C(═O)C(R⁴)(R⁵);
R⁴ is H, C₁₋₈ alkyl, C₃₋₇ cycloalkyl, halogen or phenyl, wherein the C₁₋₈ alkyl, C₃₋₇ cycloalkyl or phenyl are optionally substituted with halogen, C₁₋₄ alkyl or hydroxy;
R⁵ is H, C₁₋₈ alkyl, C₃₋₇ cycloalkyl, halogen or phenyl, wherein the C₁₋₈ alkyl, C₃₋₇ cycloalkyl or phenyl are optionally substituted with halogen, C₁₋₄ alkyl or hydroxy;
a is 0 or 1; and
b is 0 or 1.
[48] The pharmaceutical composition according to [46], wherein:
X is N;
Y is N;
Z is N;
Q is N;
R¹ is independently H, C₁₋₄ alkyl or halogen;
R³ is independently H, C₁₋₄ alkyl or halogen;
R⁹ is phenyl substituted with C₁₋₈ alkyl, which is optionally substituted with C₁₋₈ alkyl, C₁₋₈ alkoxy or halogen;
R¹⁰ is C(═O)N(R⁴)(R⁵) or C(═O)C(R⁴)(R⁵);
R⁴ is H, C₁₋₈ alkyl or halogen;
R⁵ is H, C₁₋₈ alkyl or halogen;
a is 0 or 1; and
b is 0 or 1.
[49] The pharmaceutical composition according to [46], wherein the compound of formula (IV) is compound WMA-RV4,

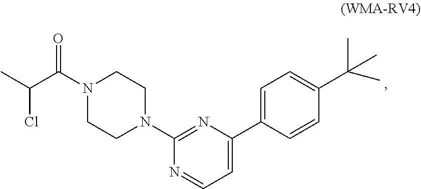 [SMILES: CC(Cl)C(=O)N1CCN(c2nccc(-c3ccc(C(C)(C)C)cc3)n2)CC1]

or a salt thereof.
[50] A pharmaceutical composition comprising:
   a compound of formula (V),

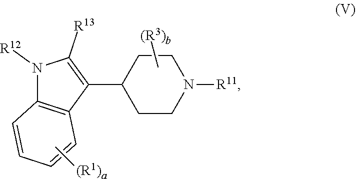

or a salt thereof,
wherein:
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R¹¹ is -C₁₋₄alkyl-S(O)ₙ—N(R⁴)(R⁵)—R¹⁴, —S(O)ₙ—C₁₋₄alkyl—N(R⁴)(R⁵) -R¹⁴, - S(O)ₙ—C₁₋₄alkyl-C(═O)C(R⁴)(R⁵)- R¹⁴, S(O)ₙ—C(═O)C(R⁴)(R⁵)- R^{14, _S(O)}ₙ—R¹⁴, —S(O)ₙ—C₁₋₄alkyl-R¹⁴ or —C₁₋₄4alkyl-S(O)ₙ—C₁₋₄alkyl-R¹⁴;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle or 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl;
R¹² is H, C₁₋₈ alkyl, C₃₋₇ cycloalkyl, halogen or C₆₋₁₀ aryl, wherein the C₁₋₈ alkyl, C₃₋₇ cycloalkyl C₆₋₁₀ aryl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R¹³ is H, C₁₋₈ alkyl or halogen;
a is 0, 1, 2, 3 or 4;
b is 0, 1, 2, 3 or 4;
m is 1 or 2; and
n is 1 or 2; and
   a pharmaceutically acceptable carrier.
[51] The pharmaceutical composition according to [50], wherein:
R¹ is C₁₋₄ alkyl;
R¹² is H or C₁₋₄ alkyl;
R¹³ is H or C₁₋₄ alkyl;
R¹¹ i s —S(O)ₙ—R¹⁴, —S(O)ₙ—C₁₋₄alkyl-R¹⁴ or —C₁₋₄alkyl-S(O)ₙ—C₁₋₄alkyl-R¹⁴;
R¹⁴ is C₆₋₁₀ aryl or 5- to 6-membered heteroaryl, wherein the C₆₋₁₀ aryl and 5- to 6-membered heteroaryl are optionally substituted with substituent(s) independently selected from the group consisting of halogen, C₁₋₄ alkyl, hydroxy, C₁₋₆ alkoxy, cyano, amino and phenyl;
a is 1, 2 or 3;
b is 0; and
n is 2.
[52] The pharmaceutical composition according to [50], wherein the compound of formula (V) is a compound of formula (Va),

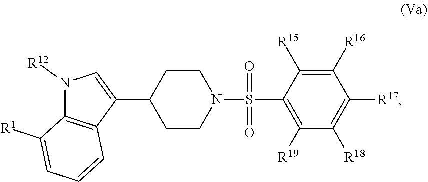

or a salt thereof,
wherein:
R¹ is C₁₋₄ alkyl;
R¹² is H or C₁₋₄ alkyl;
R¹⁵, R¹⁶, R¹⁷, R¹⁸ and R¹⁹ are each independently H, C₁₋₆ alkyl, halogen, hydroxy, C₁₋₆ alkoxy, cyano, amino or phenyl.
[53] The pharmaceutical composition according to [50], wherein the compound of formula (V) is compound WMA-RVS,

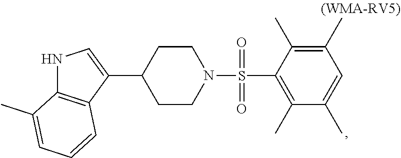

or a salt thereof.
[54] A pharmaceutical composition comprising:
   a compound of formula (VI),

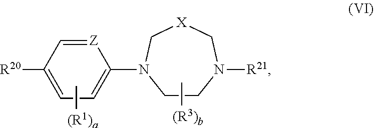

or a salt thereof,
wherein:
X is C(R²²)(R²³) or C(═O);
Z is C(R²⁴) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R²⁰ is C(═O)N(R⁴)(R⁵), C(═O)C(R⁴)(R⁵), C₁₋₄alkylC(═O)—N(R⁴)(R⁵), N(R⁴) c(═O)—R¹⁴ or N(R⁴)C(═O)-C₁₋₄alkyl-R¹⁴ , wherein the C₁₋₄alkyl is optionally substituted with halogen, C₁₋₄ alkyl or C₁₋₄ alkoxy;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and phenyl;
R²¹ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
R²² is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²³ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄ alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
a is 0, 1, 2 or 3;
b is 0, 1, 2, 3 or 4; and
m is 1 or 2; and
   a pharmaceutically acceptable carrier.
[55] The pharmaceutical composition according to [54], wherein:
X is C(R²²)(R²³);
Z is N;
R²⁰ is N(R⁴)C(═O)—R¹⁴ or N(R⁴)C(═O)-C₁₋₄alkyl-R¹⁴;
R⁴ is H or C₁₋₄ alkyl;
R¹⁴ is C₆₋₁₀ aryl or 5- to 10-membered heteroaryl, wherein the C₆₋₁₀ aryl and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and amino;
R²¹ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
b is 0 or 1.
[56] The pharmaceutical composition according to [54], wherein:
X is C(R²²)(R²³);
R²² is H;
R²³ is H:
Z is N;
R²⁰ is N(R⁴)C(═)—R¹⁴;
R⁴ is H;
R¹⁴ is phenyl, which is optionally substituted with substituent(s) independently selected from the group consisting of halogen, C₁₋₄ alkyl, hydroxy, and C₁₋₄ alkoxy; and
R²¹ is C₁ alkyl substituted with phenyl, wherein the phenyl is optionally substituted with substituent(s) independently selected from the group consisting of halogen, C₁₋₄ alkyl, hydroxy, and C₁₋₄ alkoxy.
[57] The pharmaceutical composition according to [54], wherein the compound of formula (VI) is compound WMA-RV6,

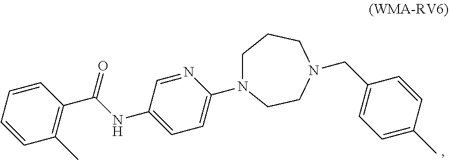 [SMILES: Cc1ccc(CN2CCCN(c3ccc(NC(=O)c4ccccc4C)cn3)CC2)cc1]

or a salt thereof.
[58] A pharmaceutical composition comprising:
   a compound of formula (VII),

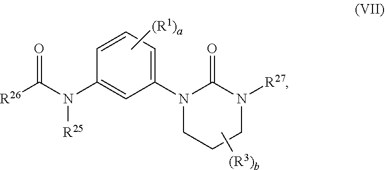

or a salt thereof,
wherein:
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl,
R²⁵ is H, C₁₋₈ alkyl or C(═O)C(R⁴)(R⁵);
R²⁶ is H, C₁₋₈ alkyl, C₃₋₇ cycloalkyl, C₆₋₁₀ aryl, 5- to 10-membered heterocycle or 5- to 10-membered heteroaryl, wherein the C₁₋₈ alkyl, C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁷ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆₋₁₀ aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
a is 0, 1, 2, 3 or 4;
b is 0, 1, 2 or 3; and
m is 1 or 2; and
   a pharmaceutically acceptable carrier.
[59] The pharmaceutical composition according to [58], wherein:
R²⁵ is H or C₁₋₄ alkyl;
R²⁶ is a 5- to 6-membered heterocycle or 5- to 6-membered heteroaryl, which is optionally substituted with substituent(s) independently selected from the group consisting of
R²⁷ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
b is 0 or 1.
[60] The pharmaceutical composition according to [58], wherein:
R²⁵ is H;
R²⁶ is a 5- to 6-membered heterocycle, which is optionally substituted with halogen, C₁₋₄ alkyl, hydroxy, oxo, cyano, C₁₋₄ alkoxy or phenyl;
R²⁷ is C₁ alkyl substituted with phenyl, wherein the phenyl is optionally substituted with 1 to 5 substituents independently selected from the group consisting of halogen, C₁₋₄ alkyl, hydroxy and C₁₋₄ alkoxy;
a is 0; and
b is 0.
[61] The pharmaceutical composition according to [58], wherein the compound of formula (VII) is compound WMA-RV7,

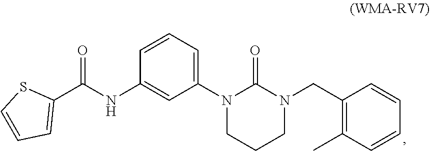 [SMILES: Cc1ccccc1CN1CCCN(c2cccc(NC(=O)c3cccs3)c2)C1=O]

or a salt thereof.
[62] A pharmaceutical composition, comprising
   a compound of formula (VIII),

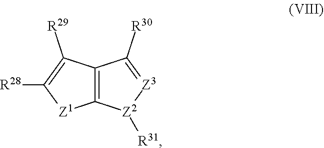

or a salt thereof,
wherein:
Z¹ is S, N or O;
Z² is S, N or O;
Z³ is S, N or O;
R²⁸ is C(═O)N(R⁴)(R⁵), C(═O)C(R⁴)(R⁵), C₁₋₄alkylC(═O)—N(R⁴)(R⁵), N(R⁴)C(═O)—R¹⁴ or N(R⁴)C(═O)-C₁₋₄alkyl-R¹⁴ , wherein the C₁₋₄alkyl is optionally substituted with halogen or C₁₋₄ alkyl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle or 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl;
R²⁹ is H, C₁₋₈ alkyl, C₃₋₇ cycloalkyl or phenyl, wherein the C₁₋₈ alkyl, C₃₋₇ cycloalkyl and phenyl are optionally substituted with halogen, C₁₋₄ alkyl, hydroxy, oxo, C₁₋₄ alkoxy, C₃₋₇ cycloalkyl or cyano;
R³⁰ is H, C₁₋₈ alkyl, C₃₋₇ cycloalkyl or C₆₋₁₀ aryl, wherein the C₁₋₈ alkyl, C₃₋₇ cycloalkyl and C₆₋₁₀ aryl are optionally substituted with halogen, C₁₋₄ alkyl, hydroxy, oxo, C₁₋₄ alkoxy, C₃₋₇ cycloalkyl or cyano; and
R³¹ is optionally substituted C₃₋₇ cycloalkyl, optionally substituted C₆₋₁₀ aryl, optionally substituted 5- to 10-membered heterocycle or optionally substituted 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and C₆₋₁₀ aryl; and a pharmaceutically acceptable carrier.
[63] The pharmaceutical composition according to [62], wherein:
Z¹ is S or O;
Z² is N;
Z³ is N;
R²⁸ is C(═O)N(R⁴)(R⁵) or C₁₋₄alkylC(═O)—N(R⁴)(R⁵), wherein the C₁₋₄alkyl is optionally substituted with halogen or C₁₋₄ alkyl;
R⁴ is H or C₁₋₄ alkyl, wherein the C₁₋₄ alkyl is optionally substituted with halogen or C₁₋₄ alkyl;
R⁵ is H or C₁₋₄ alkyl, wherein the C₁₋₄ alkyl is optionally substituted with halogen or C₁₋₄ alkyl;
R²⁹ is H or C₁₋₄ alkyl, wherein the C₁₋₄ alkyl is optionally substituted with halogen or C₁₋₄ alkyl;
R³⁰ is H or C₁₋₄ alkyl, wherein the C₁₋₄ alkyl is optionally substituted with halogen or C₁₋₄ alkyl; and
R³¹ is optionally substituted phenyl or optionally substituted 5- to 6-membered heteroaryl, wherein the phenyl and 5- to 6-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl.
[64] The pharmaceutical composition according to [62], wherein:
Z¹ is S;
Z² is N;
Z³ is N;
R²⁸ is C(═O)N(R⁴)(R⁵);
R⁴ is H;
R⁵ is C₁₋₄ alkyl, wherein the C₁₋₄ alkyl is optionally substituted with halogen or C₁₋₄ alkyl;
R²⁹ is H;
R³⁰ is C₁₋₄ alkyl, wherein the C₁₋₄ alkyl is optionally substituted with halogen or C₁₋₄ alkyl; and
R³¹ is phenyl.
[65] The pharmaceutical composition according to [62], wherein the compound of formula (VIII) is compound WMA-RV8,

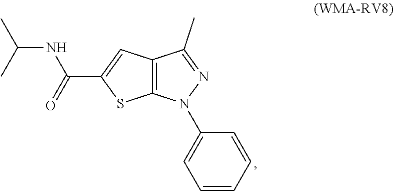 [SMILES: Cc1nn(-c2ccccc2)c2sc(C(=O)NC(C)C)cc12]

or a salt thereof.
[66] A pharmaceutical composition comprising at least one compound selected from the group consisting of:

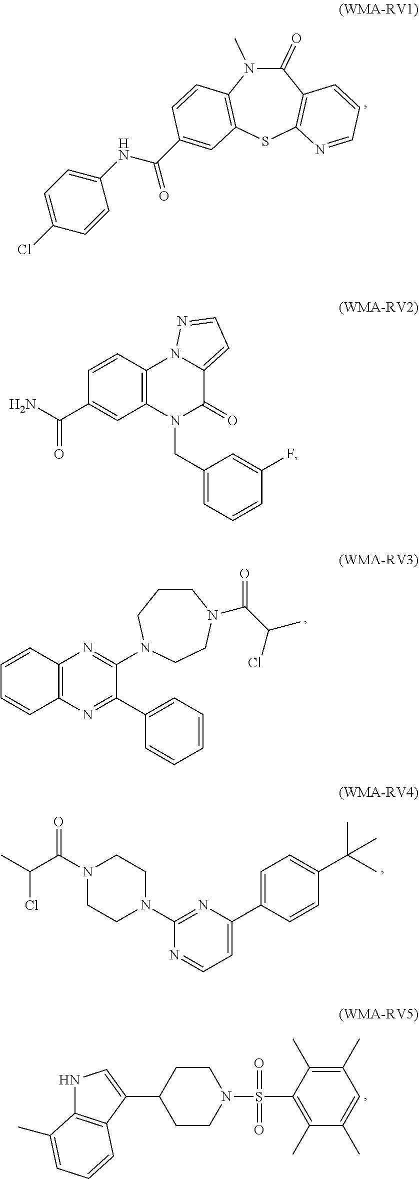

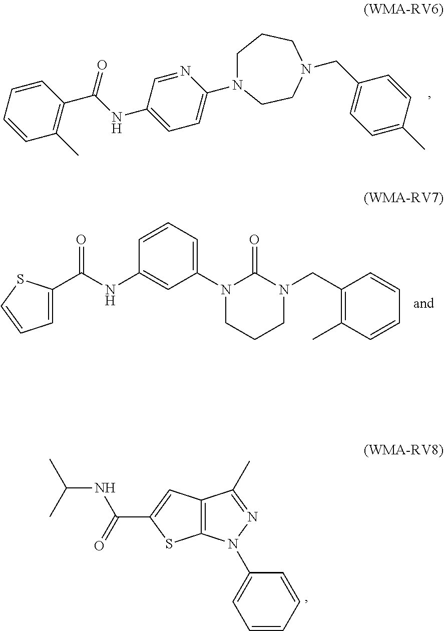

or a salt thereof,
and a pharmaceutically acceptable carrier.

### BRIEF DESCRIPTION OF THE SEVERAL VIEWS OF THE DRAWINGS

The FIGURE provides the results of a polymerase activity assay. Confluent 293T cells in 12-well plates were transfected with pCMV-T7 polymerase, pT7-M-rLuc(−) (renilla luciferase), pT7-IRES-N, pT7-IRES-L, pT7-IRES-fLuc (firefly luciferase). As controls, pT7-IRES-N and/or pT7-IRES-L was replaced with the empty pT7-IRES plasmid. Cells were incubated for 24 hours at 37° C., then treated with each tested compound, vehicle control or T-705 as a control that is an effective inhibitor of viral RNA polymerase and incubated for another 24 hours. The rLuc values normalized by fLuc values were calculated (**p≤0.01, ***p≤0.001).

### DETAILED DESCRIPTION OF THE INVENTION

A person having ordinary skill in the art will understand the terms in the present specification, claims, abstract and drawings. However, the following terms have the meanings described below.

As used herein, the number of substituents in a group when defined as “optionally substituted” or “substituted” is not particularly limited and is one or more, as long as it is substitutable. The description for each group is also applied when the group is a part of or a substituent on another group, unless specifically noted otherwise.

As used herein, “the compounds” and “the compounds of the present invention” include any compound of formula (I), (II), (III), (IV), (V), (VI), (VII) or (VIII). In more preferred embodiments, “the compounds” and “the compounds of the present invention” include any one of compounds WMA-RV1, WMA-RV2, WMA-RV3, WMA-RV4, WMA-RVS, WMA-RV6, WMA-RV7 and WMA-RV8.

Examples of a “halogen” atom as used herein include a fluorine atom, chlorine atom, bromine atom, and iodine atom. A halogen atom is preferably a fluorine atom or a chlorine atom.

The term “alkyl”, as used herein, means a straight or branched saturated monovalent hydrocarbon, including, but not limited to, one to eight carbon atoms. For example, a “C₁₋₈ alkyl group” and a “C₆ alkyl group” refer to alkyl groups with 1 to 8 and 6 carbon atoms, respectively. The same applies to other numbers. For example, methyl, ethyl, propyl, isopropyl, n-butyl, iso-butyl, sec-butyl, tert-butyl, neopentyl, hexyl, octyl, and the like.

The term “alkenyl”, as used herein, means a hydrocarbon radical having at least one double bond including, but not limited to, ethenyl, propenyl, 1-butenyl, 2-butenyl and the like. Specific examples include a vinyl group, propenyl group, methylpropenyl group, butenyl group, methylbutenyl group, pentenyl group, hexenyl group, heptenyl group, octenyl group, and the like.

The term “alkynyl”, as used herein, means a hydrocarbon radical having at least one triple bond including, but not limited to, ethynyl, propynyl, 1-butynyl, 2-butynyl and the like.

A “C₃₋₁₀ cycloalkyl group” refers to cyclic alkyl with 3 to 10 carbon atoms, including those having a partially crosslinked structure. Preferred examples of “C₃₋₁₀ cycloalkyl group” include a “C₃₋₇ cycloalkyl group”, and more preferred examples include a “C₄₋₆ cycloalkyl group”. Specific examples of “C₃₋₁₀ cycloalkyl group” include a cyclopropyl group, cyclobutyl group, cyclopentyl group, cyclohexyl group, cycloheptyl group, cyclooctyl group, adamantyl group, and the like. Specific examples of “C₃₋₇ cycloalkyl group” include a cyclopropyl group, cyclobutyl group, cyclopentyl group, cyclohexyl group, cycloheptyl group, and the like.

The term “alkoxy”, as used herein, means an 0-alkyl group wherein “alkyl” is as defined above, including, but not limited to methoxy, ethoxy, n-propoxy, iso-propoxy, n-butoxy, iso-butoxy, secondary-butoxy, tertiary-butoxy and the like.

The term “haloalkyl”, as used herein, means an alkyl radical which is substituted by a halogen atom as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl groups and the like.

The term “haloalkoxy”, as used herein, means haloalkyl-O-, including, but not limited to, fluoromethoxy, difluoromethoxy, trifluoromethoxy, 2-fluoroethoxy, 2,2-difluoroethoxy, 2,2,2-trifluoroethoxy, 2,2,2-trichloroethoxy, 3-fluoropropoxy, 4-fluorobutoxy, chloromethoxy, trichloromethoxy, iodomethoxy and bromomethoxy groups and the like.

A “C₆₋₁₀ aryl” group refers to an aromatic hydrocarbon group with 6 to 10 carbon atoms. Specific examples of “C₆₋₁₀ aryl” include a phenyl, 1-naphthyl, 2-naphthyl, and the like. Particularly preferred examples include phenyl.

A “5- to 10-membered heteroaryl group” comprises 1 to 4 atoms independently selected from the group consisting of a nitrogen atom, an oxygen atom, and a sulfur atom and includes a monocyclic 5- to 7-membered aromatic heterocyclic group (“5- to 7-membered heteroaryl group”) and bicyclic 8- to 10-membered aromatic heterocyclic group (“8- to 10-membered heteroaryl group”). A “5- to 10-membered heteroaryl group” is preferably a monocyclic 5- to 7-membered aromatic heterocyclic group (“5- to 7-membered heteroaryl group”), and more preferably a 5- to 6-membered monocyclic aromatic heterocyclic group (“5- to 6-membered heteroaryl group”).

Specific examples of “5- to 10-membered heteroaryl group” include a pyridyl group, pyridazinyl group, isothiazolyl group, pyrrolyl group, furyl group, thienyl group, thiazolyl group, imidazolyl group, pyrimidinyl group, thiadiazolyl group, pyrazolyl group, oxazolyl group, isoxazolyl group, pyrazinyl group, triazinyl group, triazolyl group, imidazolidinyl group, oxadiazolyl group, triazolyl group, tetrazolyl group, indolyl group, indazolyl group, quinolyl group, isoquinolyl group, benzofuranyl group, benzothienyl group, benzoxazolyl group, benzothiazolyl group, benzisoxazolyl group, benzisothiazolyl group benzotriazolyl group, benzimidazolyl group, 6,11-dihydrodibenzo[b,e]thiepinyl group, and the like. Preferably, the group is a pyridyl group, pyrimidinyl group, quinolyl group, or isoquinolyl group, and more preferably a pyridyl group.

A “5- to 10-membered heterocycle” includes a saturated or partially unsaturated monocyclic or polycyclic heterocyclic group comprising one or more atoms independently selected from the group consisting of a nitrogen atom, an oxygen atom, and a sulfur atom. Preferably, the 55- to 10-membered heterocycR¹ is a saturated group. The 5- to 10-membered heterocycle also includes heterocyclic groups substituted with an oxo group and heterocyclic groups having a crosslinked structure.

Specific examples of 5- to 10-membered heterocycles include the following groups: tetrahydrofuranyl group, dihydrofuranyl group, dioxolanyl group, tetrahydropyranyl group, tetrahydropyranyl group, dioxanyl group, oxathianyl group, tetrahydropyranyl group, dihydropyranyl group, pyranyl group, oxathiane dioxanyl group, dihydropyranyl group, oxadinanyl group, morpholinyl group, morpholinonyl group, oxepanyl group, dioxepanyl group, oxathiepanyl group, oxathiepanonyl group, oxazepanyl group, oxazepanonyl group, oxabicyclooctanyl group, oxabicycloheptanyl group, oxaazabicyclooctanyl group, dioxaspirononanyl group, octahydropyranopyridinyl group, pyrrolidinyl group, pyrrolidinolyl group, imidazolidinonyl group, oxazolidinonyl group, piperidinyl group, piperidinonyl group, thiomorpholinyl group, dihydropyridinonyl group, pyridinonyl group, thiomorpholinedioxinyl group, dihydropyridinyl group, dihydropyrimidinonyl group, piperazinyl group, piperazinonyl group, azepanyl group, and the like.

Examples of a “salt” of the compounds, such as a “pharmaceutically acceptable salt” include acid addition salts and base addition salts. Examples of acid addition salts include inorganic acid salts such as hydrochloride, hydrobromide, sulfate, hydroiodide, nitrate, and phosphate, and organic acid salts such as citrate, oxalate, phthalate, fumarate, maleate, succinate, malate, acetate, formate, propionate, benzoate, trifluoroacetate, methanesulfonate, benzenesulfonate, p-toluenesulfonate, and camphorsulfonate. Examples of base addition salts include inorganic base salts such as sodium salt, potassium salt, calcium salt, magnesium salt, barium salt, and aluminum salt, and organic base salts such as trimethylamine, triethylamine, pyridine, picoline, 2,6-lutidine, ethanolamine, diethanolamine, triethanolamine, tromethamine [tris(hydroxymethyl)methylamine], tert-butylamine, cyclohexylamine, dicyclohexylamine, and N-N-dibenzylethylamine. Furthermore, examples of pharmaceutically acceptable salt include salts of a basic or acidic amino acid such as arginine, lysine, ornithine, aspartic acid, and glutamic acid.

Suitable salts and pharmaceutically acceptable salts of starting compounds and target compounds are conventional nontoxic salts. Examples thereof include acid addition salts such as organic acid salts (e.g., acetate, trifluoroacetate, maleate, fumarate, citrate, tartrate, methanesulfonate, benzenesulfonate, formate, para-toluenesulfonate, and the like) and inorganic acid salts (e.g., hydrochloride, hydrobromide, hydroiodide, sulfate, nitrate, phosphate, and the like), salts with an amino acid (e.g., arginine, aspartic acid, glutamic acid, or the like), metal salts such as alkali metal salts (e.g., sodium salt, potassium salt, and the like), alkali earth metal salts (e.g., calcium salt, magnesium salt, and the like), ammonium salts, organic base salts (e.g., trimethylamine salts, triethylamine salts, pyridine salts, picoline salts, dicyclohexylamine salts, N,N′-dibenzylethylene diamine salts, and the like), and the like. Moreover, such conventional nontoxic salts can be appropriately selected by those skilled in the art.

In the methods of the invention, the compounds may be administered alone or in combination with a pharmaceutically acceptable carrier by either of the above routes previously indicated, and such administration can be carried out in single or multiple doses. More particularly, the compounds can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various nontoxic organic solvents, etc. Moreover, oral pharmaceutical compositions can be suitably sweetened and/or flavored. In general, the compounds are present in such dosage forms at concentration levels ranging from 5% to 95% by weight. For oral administration, tablets containing various excipients such as microcrystalline cellulose, sodium citrate, calcium carbonate, dipotassium phosphate and glycine may be employed along with various disintegrants such as starch and preferably corn, potato or tapioca starch, alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

A “subject” herein means an animal or a human. For example, the animal can be sheep, goats, cattle, buffalo, camels, livestock, lambs, and the like. The animals can be any age, such as adults, young adults, calves, newborns, adolescents and the like. The human can be any age, such as adults, young adults, newborns, adolescents and the like.

The compounds used in the invention are useful for symptoms and/or diseases which are induced by RVFV. The compounds are expected to have the ability to cross the blood-brain barrier. In humans, for example, the compounds are useful for treating, preventing, and/or improving symptoms of cold-like symptoms accompanying fever, chill, headache, muscular or joint pain, fatigue etc., airway inflammation symptoms such as sore throat, nasal secretion, nasal congestion, cough, sputum etc., gastrointestinal symptoms such as abdominal pain, vomitus, diarrhea etc. and, further, complications accompanying secondary infection such as acute encephalopathy and pneumonia. That is to say, the compounds used in the present invention are useful for treating and/or preventing RVFV and RVF, as well as related infectious diseases.

In humans, for example, the compounds used in the present invention are effective for shortening time to alleviation of RVF symptoms. For example, they can shorten the times until cough, sore throat, headache, nasal congestion, feverishness or chills, muscular or joint pain, and fatigue are alleviated. In particular, they are useful for shortening the times until nasal congestion, muscular or joint pain, fatigue, feverishness or chills, and headache are alleviated. Further, they are useful for shortening the times until nasal congestion and muscular or joint pain are alleviated.

In animals, for example, the compounds used in the present invention are effective to reduce or eliminate the risk associate with abortions in pregnant females and to reduce or eliminate the risk of malformations of fetuses. The compounds are expected to have the ability to cross the blood-brain barrier. Further, the compounds can, for example, reduce fever, inhibit weight loss, increase anti-bodies, and increase immune response.

A “pharmaceutically effective amount” refers to an amount that is effective for treating or preventing RVF as noted through clinical testing and evaluation, patient observation, and/or the like. An “effective amount” can further designate an amount that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition.

Additional features and advantages of the invention may be apparent from the following detailed description, examples and claims. Although particular embodiments of the invention have been described, various other known or usual changes and modifications in this field fall into the invention and are within the claims. The invention also includes the equivalents, changes, uses, or variations, which are within the spirit of the invention.

### EXAMPLES

### Example 1

EC₅₀ and CC₅₀ of selected compounds.

A plaque reduction assay was performed to determine EC₅₀ of each identified compound.

Vero E6 cells were infected with the MP12 virus, then were treated with the selected compounds (i.e., compounds WMA-RV1, WMA-RV2, WMA-RV3, WMA-RV4, WMA-RVS, WMA-RV6, WMA-RV7 and WMA-RV8) at 0μm, 0.1 μm, 1μm, 5μm, 10 μm, 20 μm, 40 μm and 80 μm, and the numbers of plaque were counted and analyzed. The half maximum cytotoxity 20 concentration (CC₅₀) of each selected compound was determined on Vero E6 cells that were treated with each compound at 0 μm, 20 μm, 40 μm, 80 μm, 160 μm, 320 μm, 640 μm, or 1280 μm for 48 hours. The cell viabilities were determined using the CellTiter-Glo® Luminescent Cell Viability Assay (Promega) following the manufacture's instruction. The results are shown in Table 1.

TABLE 1
EC₅₀ and CC₅₀ of 8 identified compounds

| Compound ID | EC₅₀ (μM) | CC₅₀ (μM) |
|---|---|---|
| WMA-RV1 | 0.8625 | 319.7 |
| WMA-RV2 | 6.962 | >1280 |
| WMA-RV3 | 2.080 | 363.2 |
| WMA-RV4 | 3.194 | 167.9 |
| WMA-RV5 | 9.142 | >1280 |
| WMA-RV6 | 1.034 | 692.1 |
| WMA-RV7 | 15.490 | >1280 |
| WMA-RV8 | 12.970 | >1280 |

### Example 2

A polymerase activity assay.

Confluent 293T cells in 12-well plates were transfected with pCMV-T7 polymerase, pT7-M-rLuc(−) (renilla luciferase), pT7-IRES-N, pT7-IRES-L, pT7-IRES-fLuc (firefly luciferase). As controls, pT7-IRES-N and/or pT7-IRES-L was replaced with the empty pT7-IRES plasmid. Cells were incubated for 24 hours at 37° C., then treated with each tested compound (i.e., compounds WMA-RV1, WMA-RV2, WMA-RV3, WMA-RV4, WMA-RVS, WMA-RV6, WMA-RV7 and WMA-RV8), vehicle control or T-705 as a control that is an effective inhibitor of viral RNA polymerase and incubated for another 24 hours. The rLuc values normalized by fLuc values were calculated (**p≤0.01, ***p≤0.001). The results are shown in the Figure.

While the invention has been described with reference to certain particular embodiments thereof, those skilled in the art will appreciate that various modifications may be made without departing from the spirit and scope of the invention. The scope of the appended claims is not to be limited to the specific embodiments described.

1. A method of treating and/or preventing Rift Valley fever, comprising administering an effective amount of a compound of formula (I), 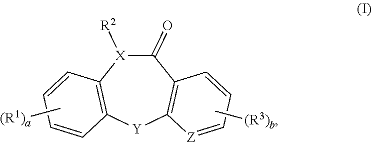
or a salt thereof,
wherein:
X is S, N and O;
Y is S, N and O;
Z is C(R^{3a}) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R² is H, C₁₋₈ alkyl, C₂₋₈ alkenyl, C₂₋₈ alkynyl, C₃₋₇ cycloalkyl, C₁₋₈ alkoxy, halogen, or halo-substituted C₁₋₈ alkoxy, wherein the C₁₋₈ alkyl, C₂₋₈ alkenyl and C₂₋₈ alkynyl are optionally substituted with halogen, C₁₋₈ alkyl, hydroxy, oxo, C₁₋₈ alkoxy, C₃₋₇ cycloalkyl, cyano or phenyl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R^{3a} H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 1, 2, 3 or 4;
b is 0, 1, 2 or 3; and
m is 1 or 2;
to a subject in need thereof.

2. The method according to claim 1, wherein:
X is N;
Y is S or O;
Z is N;
R¹ is independently H, C₁₋₈ alkyl, C(═O)N(R⁴)(R⁵) or C(═O)C(R⁴)(R⁵), wherein the C₁₋₈ alkyl is optionally substituted with substituent(s) independently selected from the group consisting of halogen, C₁₋₄ alkyl and hydroxy;
R⁴ is H or C₁₋₆ alkyl; and
R⁵ is C₁₋₆ alkyl, C₃₋₇ cycloalkyl, phenyl or 5- to 6-membered heteroaryl, wherein the C₁₋₆ alkyl, C₃₋₇ cycloalkyl, phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl.

3. The method according to claim 1, wherein:
X is N;
Y is S;
Z is N;
R¹ is C(═O)NR⁴R⁵;
R⁴ is H or C₁₋₄ alkyl;
R⁵ is phenyl or 5- to 6-membered heteroaryl, wherein the phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl;
a is 1; and
b is 0.

4. The method according to claim 1, wherein the compound of formula (I) is compound WMA-RV1, 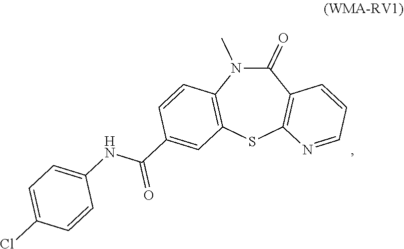 [SMILES: CN1C(=O)c2cccnc2Sc2cc(C(=O)Nc3ccc(Cl)cc3)ccc21]
or a salt thereof.

5. A method of treating and/or preventing Rift Valley fever, comprising administering an effective amount of a compound of formula (VI), 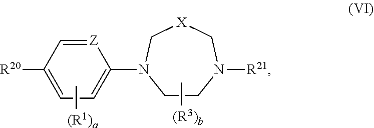
or a salt thereof,
wherein:
X is C(R²²)(R²³) or C(═O);
Z is C(R²⁴) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R²⁰ is C(═O)N(R⁴)(R⁵), C(═O)C(R⁴)(R⁵), C₁₋₄alkylC(═O)—N(R⁴)(R⁵), N(R⁴) c(═O)—R¹⁴ or N(R⁴)C(═O)-C₁₋₄alkyl-R¹⁴ , wherein the C₁₋₄alkyl is optionally substituted with halogen, C₁₋₄ alkyl or C₁₋₄ alkoxy;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and phenyl;
R²¹ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆-io aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
R²² is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²³ is H, C₁₋₈ alkyl, C₂₋₈ alkenyl, C₂₋₈ alkynyl, C₃₋₇ cylcoalkyl, C₁₋₈ alkoxy, halogen, halo-substituted substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
a is 0, 1, 2 or 3;
b is 0, 1, 2, 3 or 4; and
m is 1 or 2;
to a subject in need thereof.

6. The method according to claim 5, wherein:
X is C(R²²)(R²³);
Z is N;
R²⁰ is N(R⁴)C(═O)—R¹⁴ or N(R⁴)C(═O)-C₁₋₄alkyl-R¹⁴;
R⁴ is H or C₁₋₄ alkyl;
R¹⁴ is C₆₋₁₀ aryl or 5- to 10-membered heteroaryl, wherein the C₆₋₁₀ aryl and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and amino;
R²¹ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
b is 0 or 1.

7. The method according to claim 5, wherein:
R²² is H;
R²³ is H:
Z is N;
R²⁰ is N(R⁴)C(═O)—R¹⁴;
R⁴ is H;
R¹⁴ is phenyl, which is optionally substituted with substituent(s) independently selected from the group consisting of halogen, C₁₋₄ alkyl, hydroxy, and C₁₋₄ alkoxy; and
R²¹ is C₁ alkyl substituted with phenyl, wherein the phenyl is optionally substituted with substituent(s) independently selected from the group consisting of halogen, C₁₋₄ alkyl, hydroxy, and C₁₋₄ alkoxy.

8. The method according to claim 5, wherein the compound of formula (VI) is compound WMA-RV6, 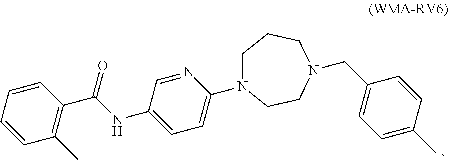 [SMILES: Cc1ccc(CN2CCCN(c3ccc(NC(=O)c4ccccc4C)cn3)CC2)cc1]
or a salt thereof.

9. A pharmaceutical composition comprising:
a compound of formula (I), 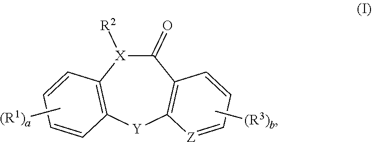
or a salt thereof,
wherein:
X is S, N and O;
Y is S, N and O;
Z is C(R^{3a}) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R² is H, C₁₋₈ alkyl, C₂₋₈ alkenyl, C₂₋₈ alkynyl, C₃₋₇ cycloalkyl, C₁₋₈ alkoxy, halogen, or halo-substituted C₁₋₈ alkoxy, wherein the C₁₋₈ alkyl, C₂₋₈ alkenyl and C₂₋₈ alkynyl are optionally substituted with halogen, C₁₋₈ alkyl, hydroxy, oxo, C₁₋₈ alkoxy, C₃₋₇ cycloalkyl, cyano or phenyl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R^{3a} is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
a is 1, 2, 3 or 4;
b is 0, 1, 2 or 3; and
m is 1 or 2; and
a pharmaceutically acceptable carrier.

10. The pharmaceutical composition according to claim 9, wherein:
X is N;
Y is S or O;
Z is N;
R¹ is independently H, C₁₋₈ alkyl, C(═O)N(R⁴)(R⁵) or C(═O)C(R⁴)(R⁵), wherein the C₁₋₈ alkyl is optionally substituted with substituent(s) independently selected from the group consisting of halogen, C₁₋₄ alkyl and hydroxy;
R⁴ is H or C₁₋₆ alkyl; and
R⁵ is C₁₋₆ alkyl, C₃₋₇ cycloalkyl, phenyl or 5- to 6-membered heteroaryl, wherein the C₁₋₆ alkyl, C₃₋₇ cycloalkyl, phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl.

11. The pharmaceutical composition according to claim 9, wherein:
X is N;
Y is S;
Z is N;
R¹ is C(═O)NR⁴ R⁵;
R⁴ is H or C₁₋₄ alkyl;
R⁵ is phenyl or 5- to 6-membered heteroaryl, wherein the phenyl and 5- to 6-membered heteroaryl are optionally substituted with substituent(s) independently selected from the group consisting of halogen, C₁₋₄ alkyl, hydroxy, C₁₋₄ alkoxy, C₃₋₇ cycloalkyl, cyano and phenyl;
a is 1; and
b is 0.

12. The pharmaceutical composition according to claim 9, wherein the compound of formula (I) is compound WMA-RV1, 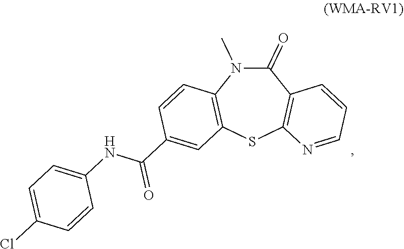 [SMILES: CN1C(=O)c2cccnc2Sc2cc(C(=O)Nc3ccc(Cl)cc3)ccc21]
or a salt thereof.

13. A pharmaceutical composition comprising:
a compound of formula (VI), 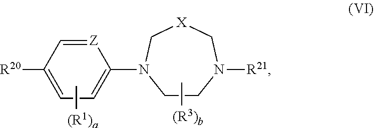
or a salt thereof,
wherein:
X is C(R²²)(R²³) or C(═O);
Z is C(R²⁴) or N;
R¹ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R³ is each independently H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with halogen, C₁₋₈ alkyl, hydroxy, oxo, C₁₋₈ alkoxy, C₃₋₇ cycloalkyl, cyano or C₆₋₁₀ aryl;
R⁵ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl, C₂₋₈ alkynyl and C₆₋₁₀ aryl are optionally substituted with substituent(s) independently selected from the group consisting of halogen, C₁₋₈ alkyl, hydroxy, oxo, C₁₋₈ alkoxy, C₃₋₇ cycloalkyl, cyano and C₆₋₁₀ aryl;
R²⁰ is C(═O)N(R⁴)(R⁵), C(═O)C(R⁴)(R⁵), C₁₋₄alkylC(═O)—N(R⁴)(R⁵), N(R⁴) c(═O)—R¹⁴ or N(R⁴)C(═O)-C₁₋₄alkyl-R¹⁴ , wherein the C₁₋₄alkyl is optionally substituted with halogen, C₁₋₄ alkyl or C₁₋₄ alkoxy;
R¹⁴ is C₃₋₇ cycloalkyl, C₆₋₁₀ aryl, 5- to 10-membered heterocycle or 5- to 10-membered heteroaryl, wherein the C₃₋₇ cycloalkyl, C₆₋₁₀ aryl, 5- to 10-membered heterocycle and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano, amino and phenyl;
R²¹ is C₁₋₈ alkyl, C₂₋₈ alkenyl, C₂₋₈ alkynyl, C₃₋₇ cycloalkyl, C₁₋₈ alkoxy, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)—N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, optionally substituted C₁₋₈ alkyl, hydroxy, oxo, optionally substituted C₁₋₈ alkoxy, optionally substituted C₃₋₇ cycloalkyl, cyano, optionally substituted C₆-io aryl and optionally substituted 5- to 10-membered heteroaryl, and wherein the optionally substituted C₁₋₈ alkyl, optionally substituted C₁₋₈ alkoxy, optionally substituted C₃₋₇ cycloalkyl, optionally substituted C₆₋₁₀ aryl and optionally substituted 5- to 10-membered heteroaryl are optionally substituted with substituent(s) independently selected from the group consisting of C₁₋₈ alkyl, C₃₋₇ cycloalkyl, C₁₋₈ alkoxy, halogen, halo-substituted C₁₋₈ alkoxy, hydroxyl, C(═O)N(R⁴)(R⁵), C(═O)C(R⁴)(R⁵), cyano, amino, oxo, mono- or di-(C₁₋₈ alkyl)amino, C₁₋₄alkylC(═O)—N(R⁴)(R⁵), C₁₋₄alkyl-S(O)ₘ—N(R⁴)(R⁵) and C₆₋₁₀ aryl;
R²² is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²³ is H, C₁₋₈ alkyl, C₂₋₈ alkenyl, C₂₋₈ alkynyl, C₃₋₇ cylcoalkyl, C₁₋₈ alkoxy, halogen, halo-substituted substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
R²⁴ is H, C₁₋₈ alkyl, C₂₋₈ alkenyl, C₂₋₈ alkynyl, C₃₋₇ cycloalkyl, C₁₋₈ alkoxy, halogen, halo-substituted C₁₋₈ alkoxy, C(═O)N(R⁴)(R⁵), C(═O)C(R⁴)(R⁵), cyano, amino, mono- or di-(C₁₋₈ alkyl)amino, C₁₋₄alkylC(═O)N(R⁴)(R⁵), C₁₋₄alkyl-S(O)ₘ—N(R⁴)(R⁵) or C₆₋₁₀ aryl, wherein the C₁₋₈ alkyl, C₂₋₈ alkenyl and C₂₋₈ alkynyl are optionally substituted with substituent(s) independently selected from the group consisting of halogen, C₁₋₄ alkyl, hydroxy, oxo, C₁₋₄ alkoxy, C₃₋₇ cycloalkyl, cyano and C₆₋₁₀ aryl;
a is 0, 1, 2 or 3;
b is 0, 1, 2, 3 or 4; and
m is 1 or 2; and
a pharmaceutically acceptable carrier.

14. The pharmaceutical composition according to claim 13, wherein:
X is C(R²²)(R²³);
Z is N;
R²⁰ is N(R⁴)C(═O)—R¹⁴ or N(R⁴)C(═O)-C₁₋₄alkyl-R¹⁴;
R⁴ is H or C₁₋₄ alkyl;
R¹⁴ is C₆₋₁₀ aryl or 5- to 10-membered heteroaryl, wherein the C₆₋₁₀ aryl and 5- to 10-membered heteroaryl are optionally substituted with substituent(s) independently selected from the group consisting of halogen, C₁₋₈ alkyl, hydroxy, oxo, C₁₋₈ alkoxy, C₃₋₇ cycloalkyl, cyano and amino;
R²¹ is C₁₋₈ alkyl substituted with phenyl, wherein the phenyl is optionally substituted with substituent(s) independently selected from the group consisting of halogen, C₁₋₄ alkyl, hydroxy, oxo, C₁₋₄ alkoxy, optionally substituted C₃₋₇ cycloalkyl, cyano, optionally substituted phenyl and optionally substituted 5- to 10-membered heteroaryl;
a is 0 or 1; and
30 b is 0 or 1.

15. The pharmaceutical composition according to claim 13, wherein:
R²² is H;
R²³ is H:
Z is N;
R²⁰ is N(R⁴)C(═O)—R¹⁴;
R⁴ is H;
R¹⁴ is phenyl, which is optionally substituted with substituent(s) independently selected from the group consisting of halogen, C₁₋₄ alkyl, hydroxy, and C₁₋₄ alkoxy; and
R²¹ is C₁ alkyl substituted with phenyl, wherein the phenyl is optionally substituted with substituent(s) independently selected from the group consisting of halogen, C₁₋₄ alkyl, hydroxy, and C₁₋₄ alkoxy.

16. The pharmaceutical composition according to claim 13, wherein the compound of formula (VI) is compound WMA-RV6, 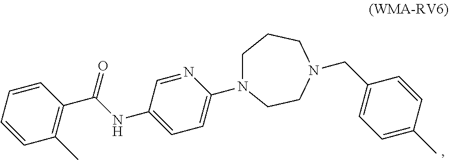 [SMILES: Cc1ccc(CN2CCCN(c3ccc(NC(=O)c4ccccc4C)cn3)CC2)cc1]
or a salt thereof.

17. A method of treating and/or preventing Rift Valley Fever, comprising administering an effective amount of at least one compound selected from the group consisting of: 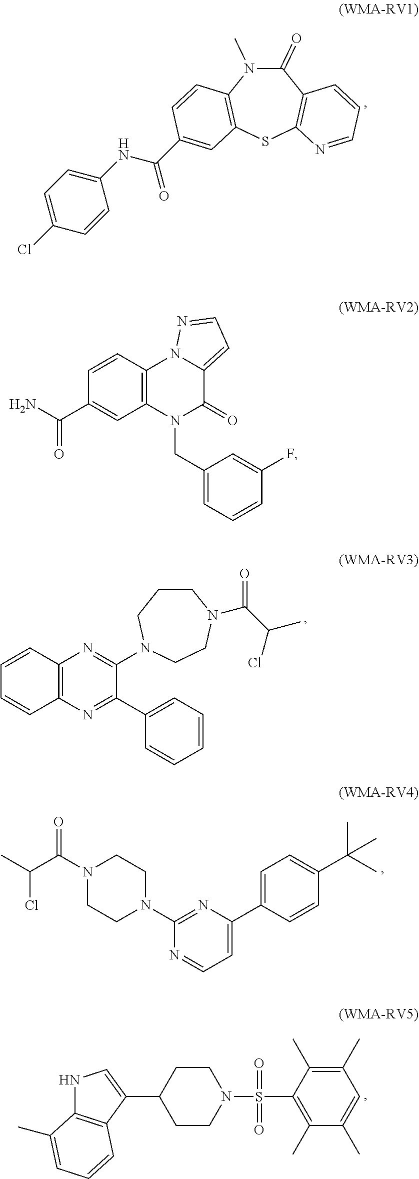 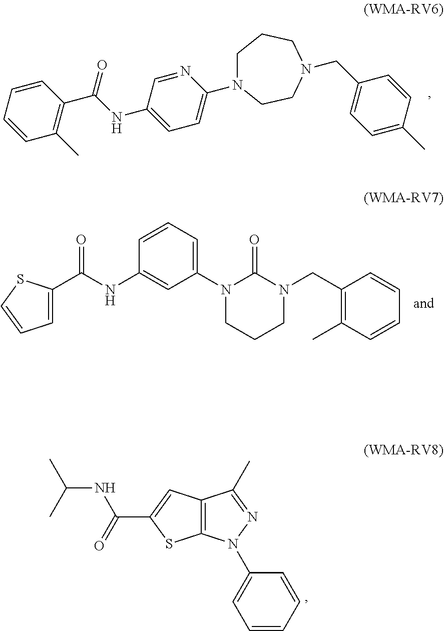
or a salt thereof,
to a subject in need thereof.

18. A pharmaceutical composition comprising at least one compound selected from the group consisting of: 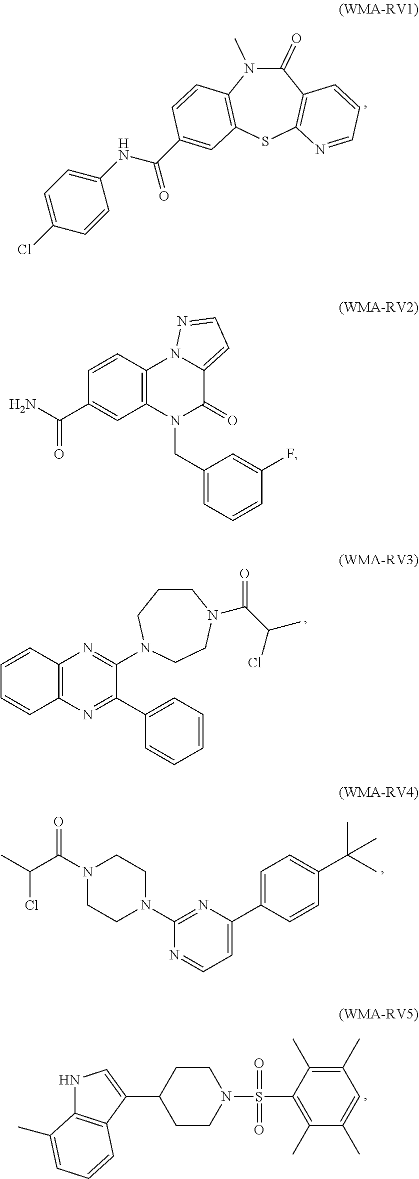 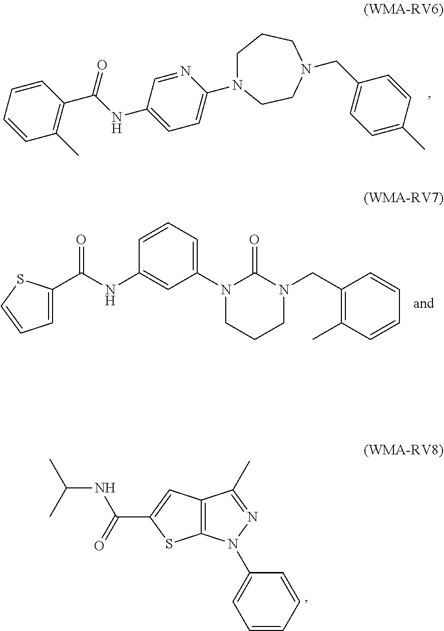
or a salt thereof,
and a pharmaceutically acceptable carrier.
